(19) United States
(12) Patent Application Publication
(10) Pub. No.: US 2002/0033180 A1
(43) Pub. Date: Mar. 21, 2002

(54) Device, an introducer and a method for providing a supplemental flow of blood

(76) Inventor: Jan Otto Solem, Stetten (CH)

Correspondence Address: BROWDY AND NEIMARK, P.L.L.C., 624 NINTH STREET, NW, SUITE 300, WASHINGTON, DC, 20001-5303, US

(21) Appl. No.: 09/950,740

(22) Filed: Sep. 13, 2001

(30) Foreign Application Priority Data
Sep. 20, 2000 (SE) 0003347-2

(51) Int. Cl. A61M 25/00; A61B 19/00

(52) U.S. Cl. 128/898; 604/284; 604/8; 606/108

(57) ABSTRACT

A device for providing a supplemental flow of blood from the left ventricle of a heart to a coronary artery thereof comprises a conduit having a first part and a second part. The first and second parts have longitudinal axes, which are at an angle to each other. The conduit further has a flexible part forming a connection between the first part and the second part.

An introducer for the device comprises a first introducing means for said first part of the conduit and a second introducing means for said second part of the conduit. The second introducing means comprises an L-shaped element for moving the second part in relation to the first part and utilizing the flexible part of the conduit in order to introduce the second part of the conduit into the coronary artery when the first part is in place.

A method for bypassing an occlusion by means of the device comprises the steps of making an incision in said coronary artery distal to the occlusion, creating a channel in a heart wall into the left ventricle cavity, inserting the first part into the channel by means of the first introducing means, inserting the second part of the conduit through said incision into the coronary artery by means of the second introducing means, and retracting said first and second introducing means.

### FIELD OF THE INVENTION

The present invention relates to a device for providing a supplemental flow of blood to a coronary artery from the left ventricle of a heart. The present invention also relates to an introducer for the device and to a method for bypassing an occlusion in a coronary artery using said device.

### BACKGROUND OF THE INVENTION

Ischemic heart disease is the most common cause of death in the Western World. Treatment is medical, interventional by means of balloon dilatation or surgery. When medical treatment is not an option any more, the next option is to treat the patient with interventional cardiology using balloons and stents to open and keep the heart arteries open. Cardiac surgery is offered as the ultimate solution.

The classical coronary artery surgery comprises bypassing the blocks in the artery causing the problem by means of conduits. The most frequent conduits are the patient's own internal mammary arteries and saphenous veins. About one million such procedures a year are conducted only in the Western World.

The surgery as such is one of the major procedures conducted on man, involving opening of the chest. In the great majority of cases the heart- and lung-machine, the so-called extra-corporeal circulation (ECC) has to be used. Much pain and morbidity results from such procedures, apart from the enormous cost thereof.

Maximal oxygenated arterial blood is available from the left atrium, the left ventricle and in the arteries of the body. Classical coronary artery bypass surgery will lead fresh arterial blood from an artery, e.g. the aorta itself, by means of a conduit to a point distal of a block or an occlusion in a coronary artery. From the aorta the blood will flow through this conduit of a saphenous vein harvested from the leg, a radial artery harvested from the forearm or the internal mammary artery harvested from inside of the chest, to the coronary artery. In some patients all suitable conduits have already been used in previous surgery.

However, the shortest distance to transport fresh arterial blood to a coronary artery is the distance from the reservoir of arterial blood in the left ventricle directly to the coronary artery through the heart muscle. In most cases this distance is less than two centimeters. It would therefore be of interest to supply the heart muscle distal to occlusions in the coronary artery with blood directly from the left ventricle cavity through the heart muscle.

U.S. Pat. No. 5,409,019 (Wilk) discloses a method for bypassing an occlusion of a coronary artery by supplying blood directly from the left ventricle cavity. According to this method a conduit is inserted by means of a catheter into the heart wall between the left ventricle cavity and the coronary artery. The method of introduction proposes that the catheter should be introduced through the blocked or occluded portion of the coronary artery. This could be quite a difficult task if the occlusion completely blocks the artery.

Furthermore, the conduit disclosed is closed during either systole or diastole to block the return flow of blood from the coronary artery to the left ventricle. The blockage of the return flow could cause areas of stagnant or turbulent blood flow. Such areas of stagnation could result in clot formation or thrombi, which could be carried to the coronary arteries causing cardiac muscle ischemia, which can be fatal. Moreover, the conduit disclosed directs blood into the coronary artery with a substantial velocity component orthogonal to the longitudinal axis of the coronary artery. This blood flow will then impinge on the coronary artery wall and could damage the wall.

U.S. Pat. No. 5,944,019 (Knudson et al) discloses another method for bypassing an occlusion of a coronary artery by supplying blood directly from the left ventricle. Here, a conduit is provided, which does not block the return flow of blood. The conduit is also curved so that the blood flow from the left ventricle is redirected inside the conduit into the coronary artery without damaging the artery wall. However, this configuration of the conduit makes it impossible to introduce the conduit by the same method as in U.S. Pat. No. 5,409,019. U.S. Pat. No. 5,944,019 proposes a few methods for introduction of the new conduit. These are complicated and involve steps that are difficult to perform. If the catheter method is used the conduit has to be introduced in two separate sections. A first step introduces a first section of the conduit into the coronary artery and a second step introduces a second section of the conduit via the left ventricle cavity into the heart wall. The sections of the conduit are introduced by means of two different catheters and a passage through the heart wall has to be matched to fit into the first section of the conduit. This matching is difficult to control, as a passage through the heart wall must be created ending exactly in the coronary artery where the first section is positioned, while the heart is moving vigorously.

Another procedure is an open chest approach. Here, an incision is made in the coronary artery and then a channel is produced from the back wall of the artery into the left ventricle cavity of the heart. Thereafter the conduit is inserted and the incision in the artery has to be sutured. This suturing is a difficult and time consuming step, especially as life-threatening bleeding might occur from the uncovered incision in the artery. Furthermore, the open chest approach is an invasive and, as such, undesired procedure.

The insertion of the conduit could also be done endoscopically, which is a much less invasive method than the open chest method. However, an endoscopic approach requires even more surgical skill, as the suturing of the incision in the artery becomes very difficult. Surgical instruments for suturing has to be introduced through trocar sheaths and be remotely manipulated. Since visualisation is limited the procedure is even more difficult. only a few very gifted surgeons may perform such endoscopic suturing.

As has been shown above, the prior art devices for providing a supplemental flow of blood to a coronary artery from the left ventricle are not easy to introduce into a patient. All devices also require complicated introducing methods, including meticulous and time-consuming suturing.

### SUMMARY OF THE INVENTION

An object of the invention is to provide a supplemental flow of blood from the left ventricle of a heart to a coronary artery by means of a device that is easy to insert into a patient. Another object of the invention is that the introduction should be possible with an endoscopic method. A further object of the invention is that the device should direct the blood into the coronary artery without damaging the vessel wall and still be easy to insert. A still further object is that no suturing at all should be necessary.

These objects of the invention are achieved by a device according to claim 1, an introducer according to claim 18 and a method according to claim 24. Advantageous embodiments are described in claims 2-17, 19-23 and 25-30.

Thus, a device is provided for providing supplemental flow of blood from the left ventricle of a heart to a coronary vessel in order to bypass an occlusion in said coronary vessel, said device comprising a conduit having a first part and a second part, said first and second parts having longitudinal axes which are at an angle to each other, said conduit comprising a flexible part between the first part and the second part.

As a result, the device could easily be inserted through a small incision in a coronary vessel. The first part could first be placed in a heart wall between the left ventricle cavity and the coronary vessel. As the conduit has a flexible part between the first part and the second part, the second part can be moved and tilted slightly in relation to the first part, which will ease the insertion of the second part. An axial movement of the second part and a tilt of the second part in the plane of the first and second parts enable the second part to be introduced through a minimal incision into the coronary vessel that could be substantially orthogonal to the first part.

Preferably, said conduit is expandable and contractible between two states, in which the conduit is dilated and contracted.

This implies that the insertion of the device is simplified further. The device can be inserted in a contracted state and, when put in its proper position the device may be expanded to form a conduit of the same size as the original vessel.

Suitably, the first and second parts of the conduit are separately expandable and contractible.

As a result, the expansion of the conduit when in place could be performed in two steps which could simplify the retraction of means for inserting the device. Also, different means for insertion of the first and second part of the conduit could be used.

Advantageously, the conduit comprises a stabilising part between the first and the second part, said stabilising part preventing axial movement of the first part and preventing the second part from rotating around its longitudinal axis.

Consequently, the device maintains the advantages of the flexible part and still prevents unwanted movement of the first and the second part in relation to each other. For example, if axial movement of the first part is not prevented, the first part could move into the second part of the conduit, thus blocking the conduit. The flow of blood would then be obstructed and the device would not function.

In a preferred embodiment, the stabilising part comprises several rigid rings surrounding the conduit.

As a result, the rings prevent axial movement of the first part of the conduit, as the rings would have to be compressed or extended if the first part is moved axially.

In another preferred embodiment, the stabilising part comprises a rigid coil.

This implies that the rigid coil prevents the first part of the conduit from moving axially.

In yet another preferred embodiment, the stabilising part comprises two bars on opposite sides of the conduit.

This means that the two bars prevent the first part of the conduit from moving axially. Since the two bars are arranged on opposite sides of the conduit, rotational movement of the second part of the conduit about its longitudinal axis is also prevented.

Preferably, the second part of the conduit extends beyond a connection to the first part.

As a result, the device could be inserted to extend along and beyond both ends of an incision made for introduction of the device. No blocking of blood flow is then needed proximal to the device and no suturing of the incision is needed since the cover of the second part of the device will cover the incision in the artery when the second part of the conduit is in its expanded state.

Suitably, the connection between the first part and the second part is closer to one end of the second part than to the other end.

This implies that the device is asymmetrical. This is advantageous for introduction of the second part of the conduit into the coronary vessel. The longer portion of the second part could first be introduced into the vessel and the shorter portion could then be introduced by retrograde movement of the second part of the conduit.

Advantageously, the first part comprises means for anchoring the device inside the left ventricle cavity of the heart.

Thus, the device could be anchored in order to stay in place when it has been introduced.

In a preferred embodiment, the means for anchoring comprises an umbrella.

In another preferred embodiment, the means for anchoring comprises a cage.

Preferably, the device is composed of a shape memory material, e.g. Nitinol.

This implies that the device could easily be contracted and expanded for simplifying the introduction of the device.

The conduit of the device has a wall or cover that preferably is made of a synthetic material normally used for vascular grafts or conduits, most preferably extruded polytetrafluoroethylene (PTFE). This material is the outer surface of the device that is exposed to the surrounding tissue and thus is composed of a material that the body will accept.

The object of the invention is also achieved by an introducer of a device for providing supplemental flow of blood from the left ventricle of a heart to a coronary vessel, said device comprising a conduit having a first part and a second part, said first and second parts having longitudinal axes which are at an angle to each other, said conduit comprising a flexible part between the first part and the second part, comprising a first introducing means and a second introducing means being arranged for introduction of said first and second parts of the conduit, respectively, said second introducing means comprising an L-shaped element for tilting and axially moving the second part of the conduit in relation to the first part of the conduit, thus utilising the flexible part of the conduit in order to introduce the second part of the conduit when the first part is in place.

Thus, the introducer utilises the flexibility of the device in order to enable insertion through a small incision in a coronary vessel.

Preferably, the first and the second introducing means comprise restraining means for keeping the first and the second part of the conduit respectively in a contracted state, said first and second introducing means comprising releasing means for releasing the restraint on the first and the second part of the conduit respectively.

This implies that the introducer keeps the device in a contracted state that is easy to handle during the introduction. When the device is placed in its proper position the restraint is released and the introducer may be retracted through the small incision leaving the device in place to provide a supplemental flow of blood to the coronary vessel.

Suitably, the restraining means of the first introducing means comprises a restraining tube having dimensions for keeping the first part of the conduit in a contracted state, said restraining tube further having a perforation along its longitudinal axis, which perforation is breakable for releasing the first part of the conduit into an expanded state.

As a result, the introducer can easily introduce a first part of the device in a contracted state and the release of the restrain can easily be performed.

In a preferred embodiment the means for releasing the restraint of the first introducing means comprises a balloon which when filled breaks the perforation of the restraining tube. The inflation of a balloon in the channel will by the force of the balloon tear heart muscle tissue around the balloon. The tear and dislocation of heart muscle tissue by the balloon will create a wide and roomy channel big enough to house the first part of the conduit without compressing it.

This means that the release of the restrain on the first part of the conduit can be controlled from outside the patient.

Preferably, the first introducing means comprises a catheter, to which the restraining means is attached, for manoeuvring the introduction of the first introducing means.

This implies that the restraining means can easily be introduced in place and is also connected to the catheter when the restraint is released and therefore is easily retracted. Desirably, at least a part of the catheter of the first introducing means is stiff. This means that the manoeuvring of the first introducing means can be exactly controlled since the catheter is not bent or stretched when manoeuvred.

Suitably, the second introducing means comprises two L-shaped elements, said two elements being connected to form a T-shaped element, which is divisible into the two L-shaped elements for retraction of the L-shaped elements after introduction of the device.

Thus, the introducer could be used to introduce a device that is T-shaped, without further complexity of the introduction. Preferably, the restraining means of the second introducing means comprises a thread, which by being wound around the second part of the conduit restrains it to its contracted state and holds it attached to the second introducing means, and the releasing means of the second introducing means is arranged to cut the thread for release of the restraint on the second part of the conduit. This means that a simple cutting of the thread will release the second part of the conduit from the second introducing means and at the same time release the restraint on the second part of the conduit.

The object of the invention is also achieved by a method for bypassing an occlusion in a coronary vessel by providing a supplemental flow of blood from the left ventricle of a heart to said coronary vessel by means of a device comprising a conduit having a first part and a second part, said first and second parts having longitudinal axes which are at an angle to each other, said conduit comprising a flexible part between the first part and the second part, comprising the steps of making an incision in said coronary vessel distal to the occlusion, creating a channel in a heart wall into the left ventricle cavity, inserting the first part of the conduit into the channel by means of a first introducing means, tilting and axially moving the second part of the conduit in relation to the first part of the conduit by means of a second introducing means, inserting the second part of the conduit through said incision into the coronary vessel by means of the second introducing means, and retracting said first and second introducing means.

As the second part of the conduit is tilted and moved in relation to the first part of the conduit, only a small incision has to be made in the coronary vessel for the introduction of the device.

Preferably, the step of creating the channel is performed by means of a puncture needle and the method further comprises the step of introducing a guide wire through the puncture needle for leading the first introducing means to the channel in the heart wall.

This means that a guide for introduction of the device is easily created at the time of creating a channel in the heart wall.

Suitably, the method further comprises the step of moving the second part antegrade into the coronary vessel and then retrograde in the coronary vessel in order to place it properly in the vessel.

This implies that the device could be introduced to cover the whole incision and thus assuring that no suturing is needed.

Advantageously, the device is expandable and contractible between an expanded state and a contracted state and the method further comprises the step of expanding the device from the contracted state to the expanded state when the device has been properly placed in the coronary vessel.

As the device is introduced in a contracted state it is easier to handle and only small cuts have to be done in the coronary vessel and the heart wall. This means that after the introduction the incision in the vessel does not have to be sutured, which is a finicky job.

Preferably, the method further comprises the step of releasing the contraction of the first part of the conduit by inflating a balloon in the first introducing means to break a restraining means in the first introducing means.

As a result the first part of the conduit could easily be transformed from the contracted state to the expanded state when properly placed in the heart wall.

Desirably, the method further comprises releasing the contraction of the second part of the conduit by cutting a thread restraining the second part to its contracted state and attaching the second part of the conduit to the second introducing means. This means that the second part of the conduit could be released from the second introducing means and at the same time be released of the restraint by a simple cutting of a thread.

Suitably, the method further comprises the step of blocking the blood flow in the coronary vessel proximal to the second part of the conduit.

Thus, bleeding is prevented proximal to the device if the device does not cover the whole incision.

Advantageously, the puncture needle is inserted through a back wall of the vessel into the left ventricle cavity so as to create a channel from the left ventricle cavity to the coronary vessel.

This implies that the channel automatically connects the left ventricle cavity of the heart with the coronary vessel.

### BRIEF DESCRIPTION OF THE DRAWINGS

The invention will now be described in more detail by exemplification with reference to the accompanying drawings.

FIG. 1 illustrates the anatomy of a human heart.

FIG. 2 is a perspective view of a heart and a coronary artery, where the heart has been cut open in the anterior and lateral walls showing the left ventricle cavity inside the heart.

FIG. 3 is a perspective view corresponding to FIG. 2 where a channel is created between the left ventricle cavity and the coronary artery and a device is inserted into the channel and into the coronary artery.

FIGS. 4 and 5 are schematic front views of the device according to the invention.

FIG. 6 is a schematic side view of the device according to the invention.

FIG. 7 is a cross-sectional view of a first embodiment of the device according to the invention, taken along lines VII-VII in FIG. 8.

FIG. 8 is a cross-sectional view of the device in FIG. 7, taken along lines VIII-VIII in FIG. 7.

FIG. 9 is a front view of a second embodiment of the device according to the invention.

FIG. 10 is a cross-sectional view of a third embodiment of the device according to the invention, taken along lines X-X in FIG. 11.

FIG. 11 is a cross-sectional view of the device in FIG. 10, taken along lines XI-XI in FIG. 10.

FIG. 12 is a cross-sectional view of a fourth embodiment of the device according to the invention, taken along lines XII-XII in FIG. 13.

FIG. 13 is a cross-sectional view of the device in FIG. 12, taken along lines XIII-XIII in FIG. 12.

FIG. 14 is a front view of a device according to the invention having an end in umbrella form for anchoring of the device when properly placed in the heart.

FIG. 15 is a front view of a device according to the invention having a cage at an end to be inserted into the left ventricle cavity of the heart.

FIG. 16 is a front view of an introducer of the device according to the invention having a first and a second introducing means.

FIG. 17 is a cross-sectional view of the first introducing means of the introducer along lines XVII-XVII in FIG. 16.

FIG. 18 is a side view of the introducer in FIG. 16.

FIG. 19 is a side view of the introducer in FIG. 16 where the second introducing means of the introducer is being retracted after positioning of the device.

FIG. 20 is a front view of the first introducing means of the introducer in FIG. 19 where a balloon is inflated for releasing of restraint on a first part of the device.

FIG. 21 is a side view of the first introducing means of the introducer in FIG. 20 being retracted after positioning of the device.

FIG. 22 is a cross-sectional view of the heart where a puncture needle is put through the back wall of the coronary artery, through the left ventricle wall and into the left ventricle cavity.

FIG. 23 is a cross-sectional view of the channel in the heart wall, where a guide wire is led through the puncture needle into the left ventricle cavity.

FIG. 24 is a sectional view of the channel in the heart wall and the coronary artery where the introducer is positioned with the device in the channel in the left ventricle wall and in the coronary artery.

FIG. 25 is a sectional view of the device in the channel in the heart wall and the coronary artery where the second introducing means of the introducer has been retracted.

FIG. 26 is a sectional view of the device in the channel in the heart wall and the coronary artery where a syringe is being used to inflate the balloon in the first introducing means.

FIG. 27 is a sectional view of the device in the channel in the heart wall and the coronary artery where the first introducing means of the introducer has been retracted as well.

FIG. 28 is a cross-sectional view of the device in the channel in the heart wall.

FIG. 29 is a cross-sectional view of the left ventricle of the heart where three different devices are positioned in three coronary arteries illustrating different means for anchoring the device in the left ventricle cavity.

FIG. 30 is a cross-sectional view of the left ventricle of the heart where a channel has been made in the heart wall at a distance away from the coronary artery and a device is placed connecting the coronary artery with the left ventricle cavity.

FIG. 31 is a perspective view of the left ventricle of the heart where another embodiment of the device has been introduced into the channel in the heart wall and the coronary artery.

FIG. 32 is a front view of a second embodiment of the introducer of the device for introducing the device shown in FIG. 31.

### DETAILED DESCRIPTION OF A PREFERRED EMBODIMENT

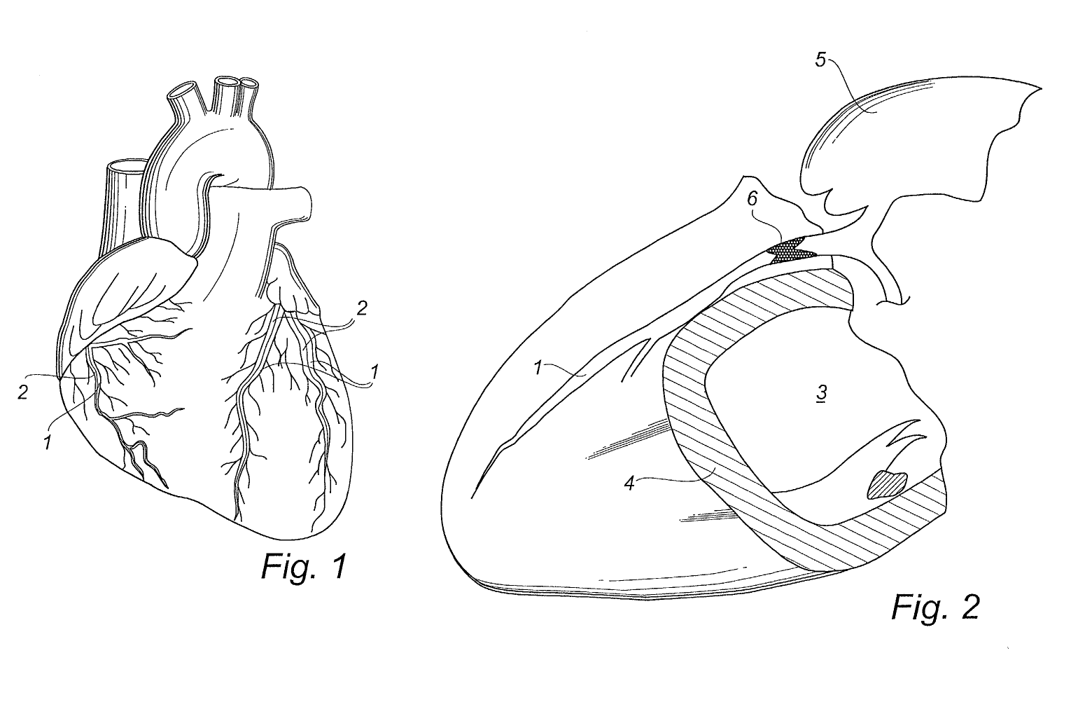

Referring to FIG. 1, the front anatomy of a heart relevant to the present invention is shown. Coronary arteries 1, which supply the heart muscle with blood, and coronary veins 2 extending substantially along the coronary arteries, are shown.

In FIG. 2, a left ventricle 3 of the heart is shown. The left ventricle 3 being a cavity inside the heart is shown with a part of an anterior wall 4 of the heart cut out. The cutting edge indicates the thickness of the heart wall 4 in relation to the coronary artery 1. The coronary artery 1 has its origin in the aorta 5 and, in this case, it is the left anterior descending branch of the left coronary artery.

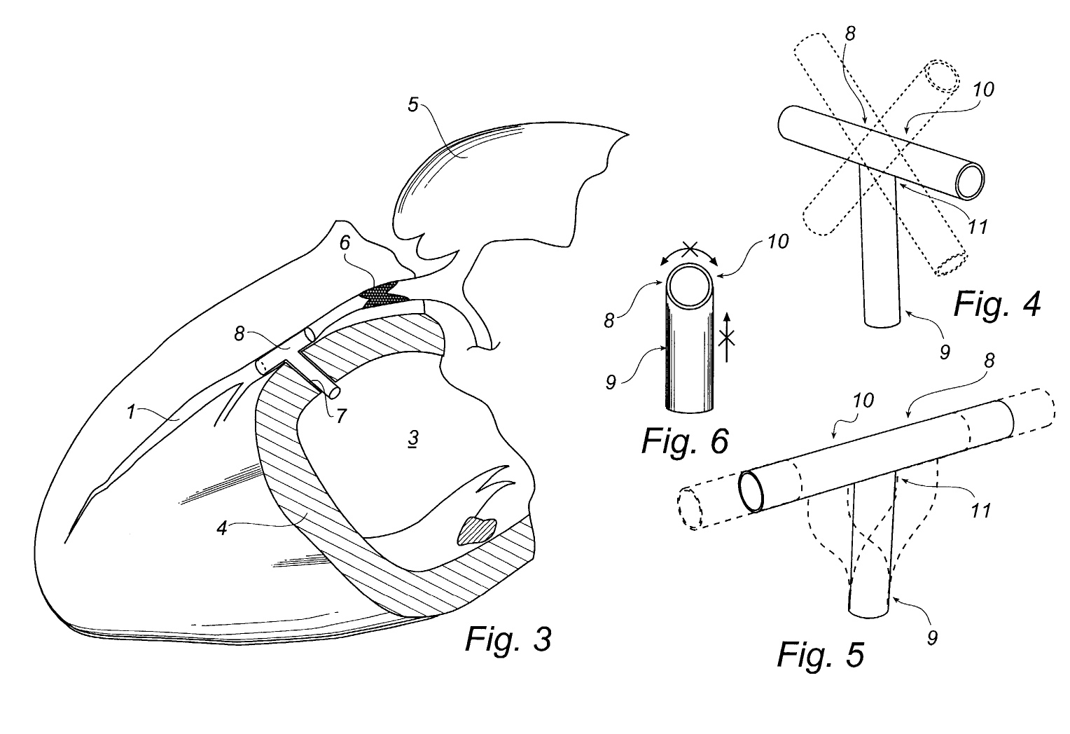

Also, an occlusion 6 is shown in the coronary artery 1. FIG. 3 shows a channel 7 made through the heart wall 4 between the left ventricle cavity 3 and the coronary artery 1. This channel 7 bypasses the occlusion 6 in the coronary artery 1 thus enabling blood flow from the left ventricle cavity 3 into the coronary artery 1 distal to the occlusion 6.

FIG. 3 also illustrates a device 8 according to the invention inserted into the channel 7 and the coronary artery 1. This device 8 connects the left ventricle cavity 3 of the heart with the coronary artery 1 and directs blood from the left ventricle cavity 3 into the coronary artery 1.

Preferred embodiments of the device 8 should have the characteristics illustrated in FIGS. 4-6.

The device 8 is hollow and thus constitutes a conduit. This conduit has a first part 9 and a second part 10, said first and second parts 9, 10 having longitudinal axes which are at an angle to each other. The first part 9 is to be placed in the channel 7 in the heart wall 4 and the second part 10 is to be placed in the coronary artery 1 distal to the occlusion 6.

In order that the device 8 should be easy to position as illustrated in FIG. 3, it should be possible to vary the angle between the longitudinal axes of the first part 9 and the second part 10. According to the invention, the first part 9 and the second part 10 are connected to each other by a flexible part 11 which, as illustrated by dotted lines in FIG. 4, allows varying of the angle between the longitudinal axes of the first part 9 and the second part 10.

Further, it is also essential that the second part 10 is movable along its longitudinal axis relative to the first part 9, as illustrated by dotted lines in FIG. 5. The flexible part 11 allows such movements.

FIG. 6 illustrates a preferred stability of the device 8 as regards movements of the first part 9 along its longitudinal axis relative to the second part 10, and also a preferred stability of the device 8 as regards rotating movements of the second part 10 about its longitudinal axis. Thus, the first part 9 should not be able to move along its longitudinal axis towards and into the second part 10, thereby eliminating the risk of a decrease in the blood flow through the device. Likewise, the second part 10 should not be able to rotate about its longitudinal axis, thereby eliminating another risk of a decrease in the blood flow through the device because of an angulation or kinking at the connection between the first and second parts 9, 10 of the conduit.

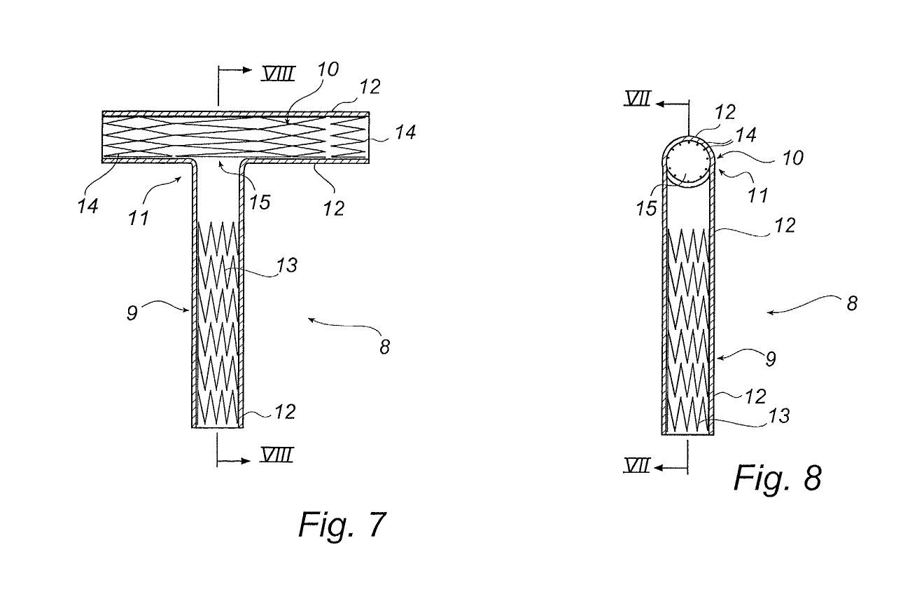

A first embodiment of the device 8 is shown in FIGS. 7 and 8. Here, the conduit of the device 8 has a wall 12 that is made of a synthetic material normally used for vascular grafts or conduits, like polyethylene, polyvinyl, polyurethane, silicon, dacron, but preferably of extruded polytetrafluoroethylene (PTFE). However, it may also be made of resorbable synthetic materials like polyglycolicacid, polyglactin, polydioxanone or any other resorbable synthetic material. The conduit also has supporting inner structures 13, 14. These are typical vascular stent structures. Preferably, there are two stents in the conduit, one stent 13 in the first part 9 and one stent 14 in the second part 10. The flexible part 11 has no inner stent structure supporting its wall 12 and thus it will allow the movements described above.

The stent 14 in the second part 10 of the conduit has an opening 15 in the stent mesh, e.g. made by a selected distribution of metal struts in the stent. This opening or hole 15 corresponds to the origin of the flexible part 11 of the conduit such that a channel is provided from the free end of the first part 9 to at least one of the free ends of the second part 10.

Further, the opening 15 is asymmetrically located on the second part 10 of the conduit, i.e. closer to one end than the other. Therefore, the second part 10 of the conduit has one long portion and one short portion. This asymmetrical location simplifies insertion of the device 8 as will be described in more detail below.

The stents 13, 14 inside the conduit are preferably made of a memory metal, for example Nitinol, being an alloy of nickel and titanium. This memory metal makes the conduit relatively flexible, compressible and bendable. It may, for instance, be compressed to a considerable degree before insertion and may, after release, return to its original T-shape inside the coronary artery and the heart muscle. Other possible materials for the stent are synthetic materials of the above mentioned types, including resorbable materials.

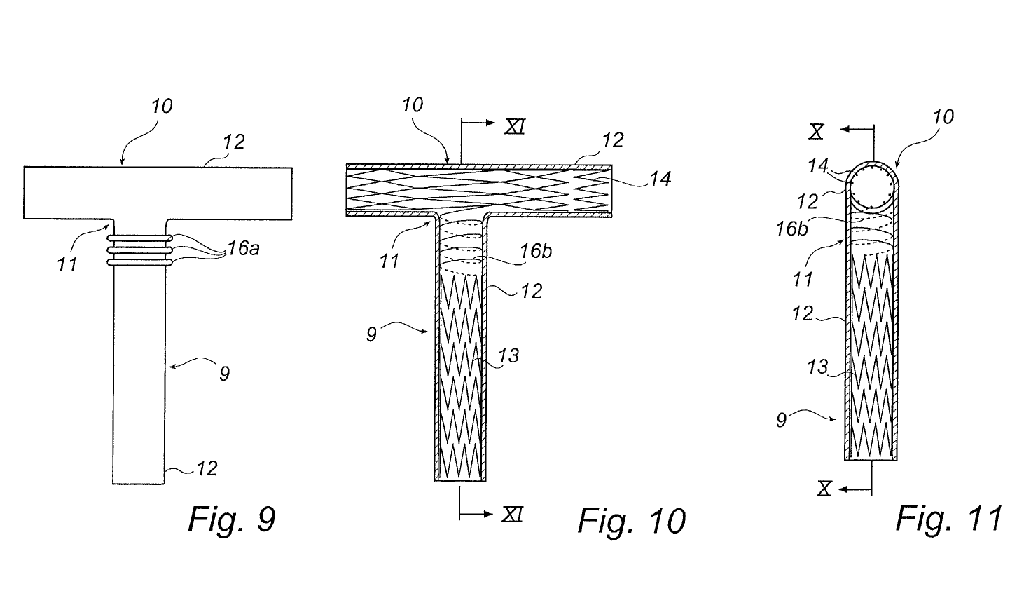

FIG. 9 illustrates a modification of the device 8 shown in FIGS. 7 and 8 in the form of a stabilising means 16. Thus, the flexible part 11 of the device 8 is strengthened by rings 16a outside or inside of the conduit wall 12. The rings 16a are made of the same material as the conduit wall 12 or any other flexible material to prevent a collapse or kinking in this area of the device 8.

In FIGS. 10 and 11, another embodiment of the stabilising means 16 is shown. Here, the stabilising means consists of a coil 16b that extends along the flexible part 11 inside or outside of the conduit wall 12.

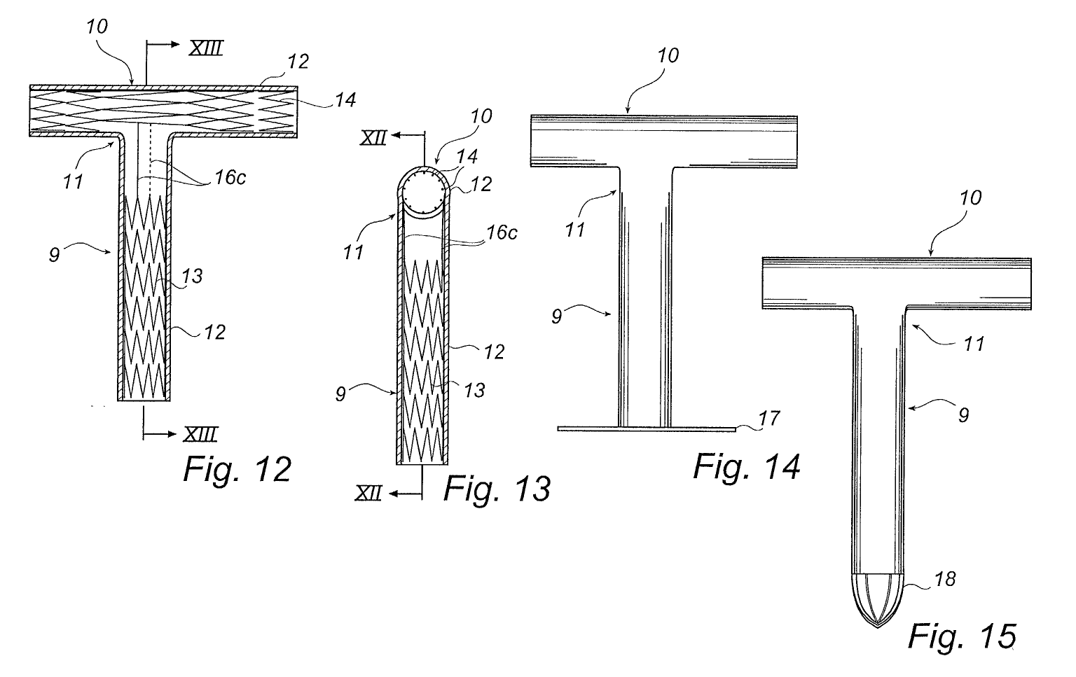

In FIGS. 12 and 13, two bars 16c are arranged on opposite sides of the conduit as stabilising means. Preferably, these bars 16c connect the stent 13 with the stent 14 close to the opening 15.

The modifications according to FIGS. 9-13 ensure the preferred stabilities referred to above and illustrated in FIG. 6.

The free end of the first part 9 of the device 8 that is to be placed inside the left ventricle cavity may have a variety of configurations. In FIG. 14 said free end has the shape of an umbrella or a fan part 17 to be placed inside the left ventricle cavity 3. The umbrella or fan 17 may be covered or may be a naked stent formation of Nitinol.

In FIG. 15 another embodiment of the device 8 is shown where the end of the first part 9 to be placed inside the left ventricle cavity 3 is a cage 18 of stent material.

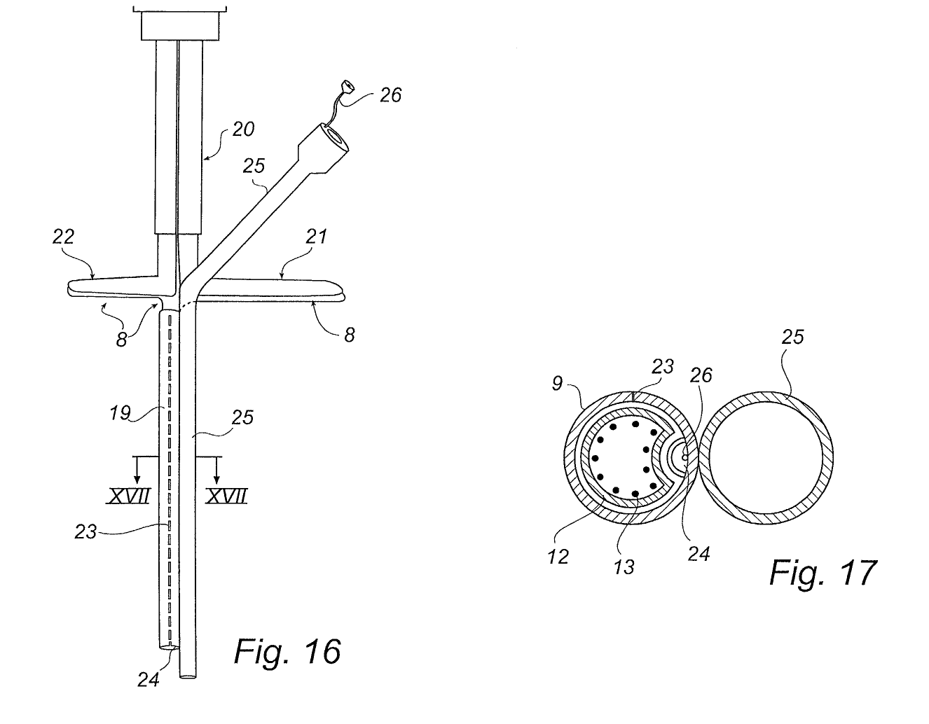

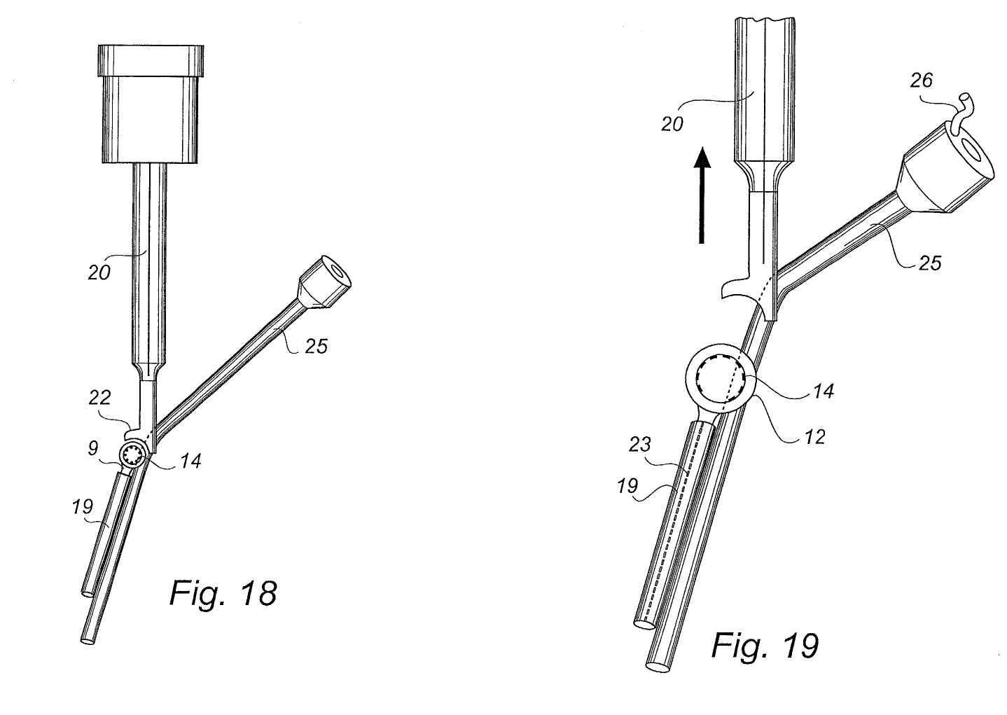

FIGS. 16-20 depict a preferred embodiment of an introducer for introduction of the device 8 into a patient. The device 8 itself is attached to the introducer. The introducer has two parts; a first introducing means comprising a restraining tube 19, and a second introducing means comprising a handle 20. The handle 20 has two arms, one long arm 21 and one short arm 22, corresponding to the long portion and the short portion of the second part 10 of the conduit, respectively. By means of a thread the second part 10 of the conduit may be restrained and compressed on the handle 20 to a very small diameter and may later be released from the handle 20 by activation of a release mechanism of the handle 20. The thread is wound around the second part 10 of the conduit, thus restraining it, and the release of the restrain is accomplished by cutting the thread. The configuration of the handle 20 and the second part 10 of the conduit with one long arm and one short arm facilitates the insertion of the device 8 through an incision in an artery. More precisely, the long arm, or the toe, is first inserted into one end of the incision opening in the vessel so far that the entire second part 10 of the conduit is inside the vessel. Then, through a reciprocal movement the second part 10 of the conduit is moved backwards so far that the short arm, or the heel, is completely inside the vessel proximal to the incision.

The restraining tube 19, shown in cross-section in FIG. 17, serves the same purpose as the handle 20. In manufacturing of the device 8, the first part 9 of the conduit is compressed to a very small diameter and inserted into the restraining tube 19 of a very small diameter. This tube 19 has a perforation line 23 from one end of the tube 19 to the other. This perforation line 23 indicates an area of low resistance along the restraining tube 19. By inflating a balloon 24 inside the restraining tube 19 but outside the first part 9 of the conduit 8 the perforation line 23 will break and the device 8 will be released. Due to the force of the memory metal in the first part 9, the first part 9 will expand and fill out the channel 7 created in the left ventricle muscle wall by the balloon 24 that during inflation would be expanded to a considerably larger size than the diameter of the first part 9 of the conduit. The restraining tube 19 itself is attached to a catheter 25. This catheter 25 is so stiff as to be able to serve as a navigational aid for finding the left ventricular cavity. It is also used for the retrieval of the restraining tube 19 from the channel 7 when the expansion of the first part 9 of the conduit is completed. A tube 26 connected to the balloon 24 inside the restraining tube 19 may also run inside the catheter 25 and is used for the inflation of the balloon 24.

In FIG. 19 the handle 20 is shown released and the second part 10 of the conduit has expanded. This release is performed when the device 8 has been positioned correctly inside the coronary artery 1.

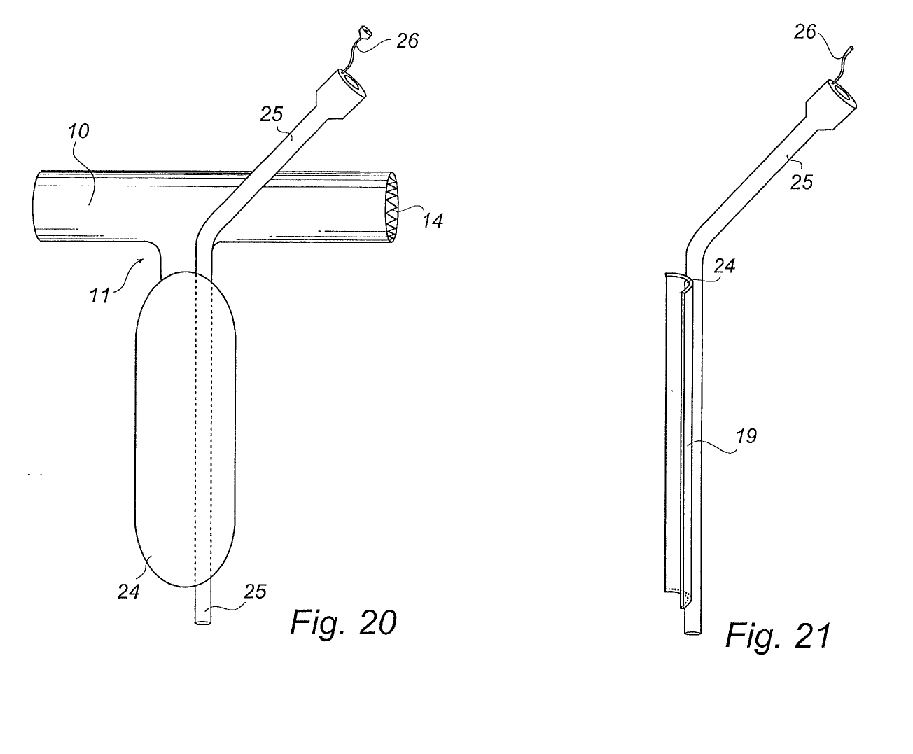

When the handle 20 has been released and retracted, the restraining tube 19 can be released. In FIG. 20 the balloon 24 is inflated, breaking the perforation line 23 of the restraining tube 19. By injecting fluid under high pressure through a tube 26 connected to the balloon 24, the balloon 24 expands inside the restraining tube 19. This forces the restraining tube 19 wide open along the perforation line 23 to release the first part 9 of the conduit inside the channel 7 in the left ventricular wall. By designing the balloon 24 much bigger than the first part 9 of the conduit one can create a channel 7 that is roomy for the first part 9. In addition, the stent 13 inside the first part 9 keeps the first part 9 open and resists any compressive force from the muscle.

Referring to FIG. 21, the split restraining tube 19 attached to the catheter 25 can be retrieved by pulling out the catheter 25.

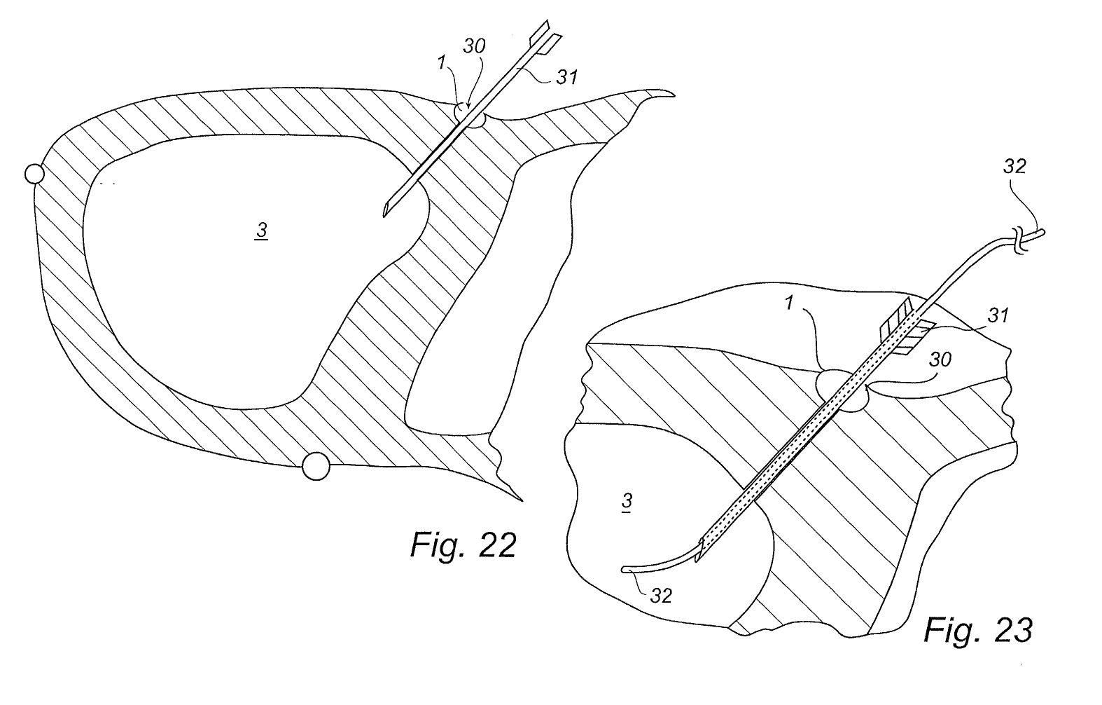

Referring to FIGS. 22-28, the operation of the device 8 will now be described in more detail.

Referring now to FIG. 22, first an incision 30 is made in the coronary artery 1 distal to an occlusion 6 in the artery 1. In the opening or incision 30 of the coronary artery 1 a puncture needle 31 is pushed through its back wall and into the left ventricle cavity 3. When fresh arterial blood is flowing out of the needle 31, the tip of the needle 31 is inside the left ventricular cavity 3.

Referring to FIG. 23, a guide-wire 32 is pushed through the needle 31 into the left ventricle cavity 3. The correct position of the needle 31 and the guide-wire 32 might be confirmed by means of ultrasound or X-ray fluoroscopy. After confirmation of the correct position the needle 31 is retracted and the wire 32 may be used for navigational purpose to direct the device 8 and especially the first part 9 of the conduit into the desired position.

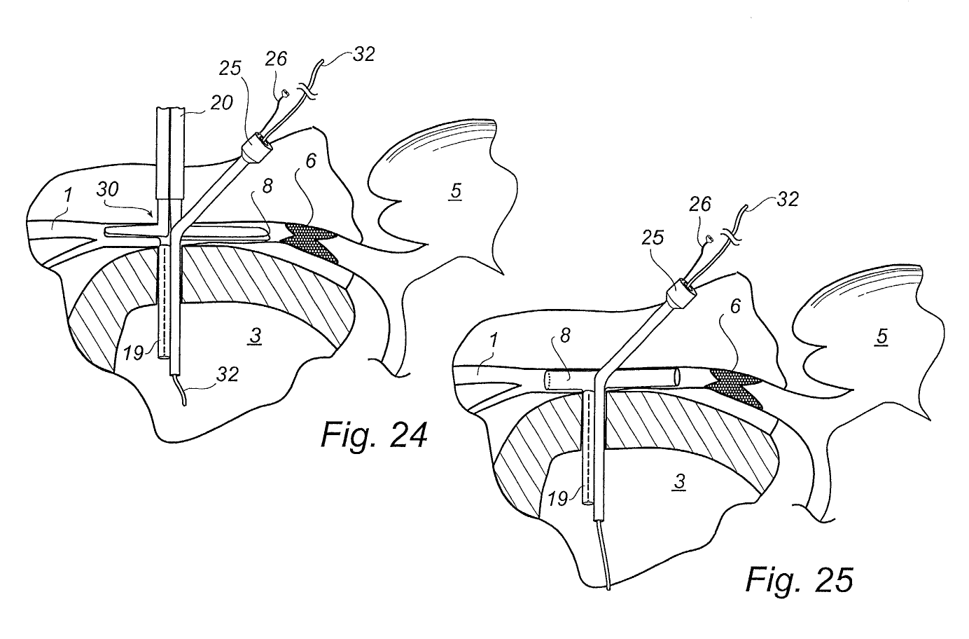

Referring to FIG. 24, the catheter 25 is inserted over the guide-wire 32 until its tip is within the left ventricular cavity 3. The guide-wire 32 is running through a lumen of the catheter 25 for the navigation of the device 8 into the right position.

The device 8 is advanced until the whole of the second part 10 of the conduit is fully inside the artery 1. The asymmetrical positioning of the handle 20 and the first part 9 of the conduit on the second part 10 of the conduit facilitates the positioning of the second part 10 since the long end is inserted first and thereafter the short end is backed into position on the other side of the opening 30 in the artery 1. The flexible part 11 of the device 8 gives the necessary freedom of movement for this positioning of the second part 10 of the conduit.

Once the second part 10 is in the correct position in the artery 1, the release mechanism of the handle 20 is activated, the second part 10 expands inside the artery 1 and the handle 20 is retrieved, cf. FIG. 25.

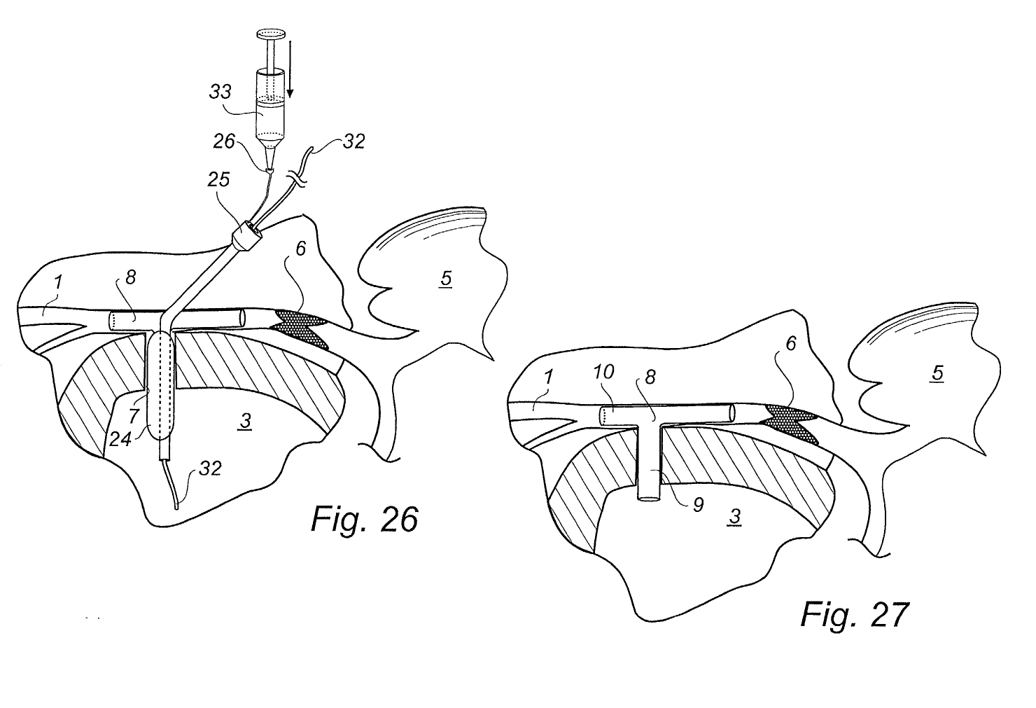

Referring to FIG. 26, a syringe 33 with fluid is connected to the small tube 26 leading to the balloon 24 located inside the restraining tube 19. The balloon 24 is next to the first part 9 of the conduit also inside the restraining tube 19. Fluid under high pressure is injected into the balloon 24, whereby the balloon 24 increases to a considerably bigger size than the first part 9 of the conduit has in its expanded state. The restraining tube 19 will flare open along the perforation line 23 and the first part 9 of the conduit is free. The restraining tube 19 now will be more like a small strip sitting on top of the catheter 25 and is pulled out from the newly created channel 7 together with the catheter 25.

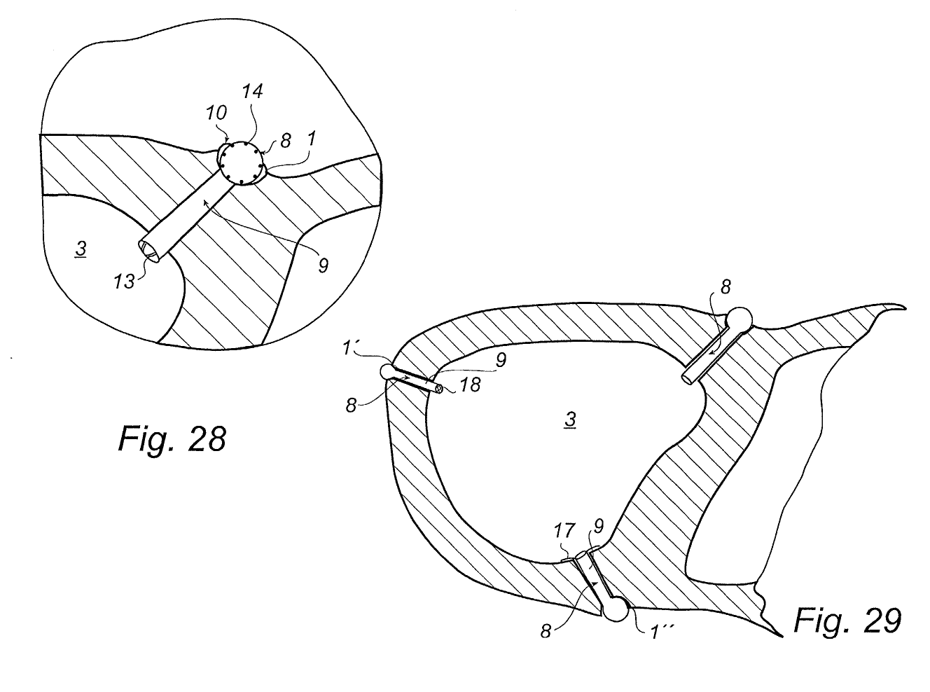

In FIGS. 27 and 28, the device 8 is shown in its correct position. The second part 10 of the conduit is in the coronary artery 1, in this case the LAD below the occlusion 6 and the first part 9 of the conduit is penetrating the heart muscle into the left ventricular cavity 3. Now fresh arterial blood may pass from the left ventricular cavity 3 directly into the coronary artery 1.

Proceeding identically as described in FIGS. 22-28, the same kind of connections may be created in the other arteries of the heart by means of the new conduit. In FIG. 29 two additional conduits are inserted. The one located in the circumflex artery 1′ has a cage 18 in the end of the first part 9 of the conduit and the one located in the posterior descending branch 1″ from the right coronary artery has an umbrella 17 at the end of the first part 9.

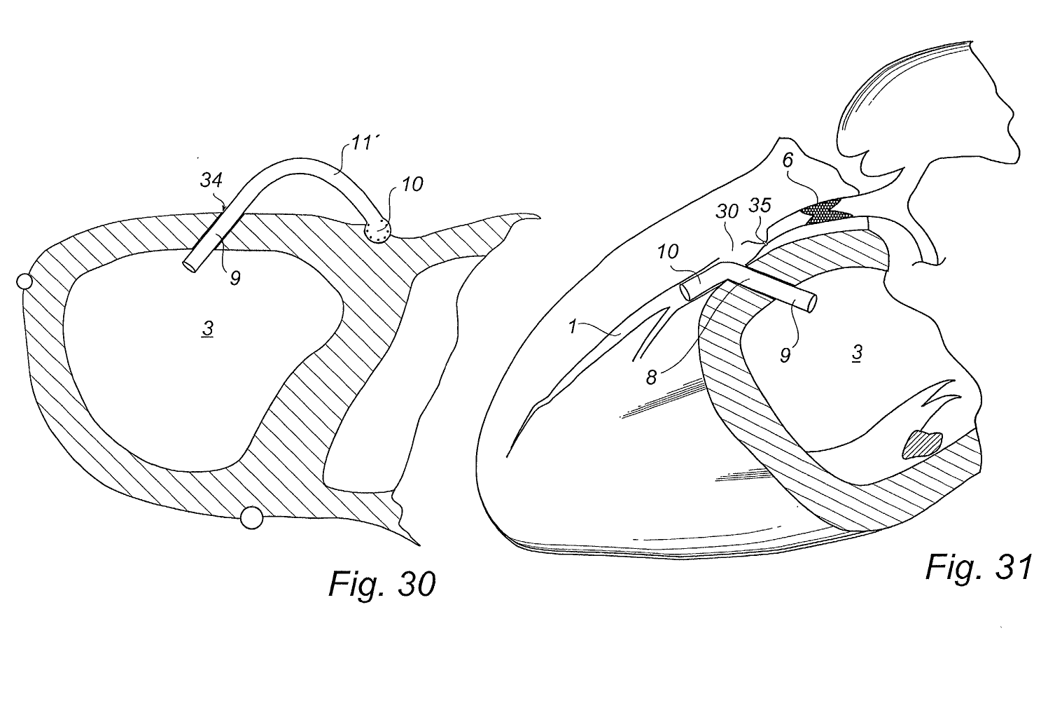

In some cases the anatomy might not be favourable for creation of a channel 7 directly from the artery 1 through the heart muscle. The new device 8 may also be used in such cases as shown in FIG. 30. The flexible part 11′ of the device 8 is extended for this purpose and the first part 9 of the conduit can then be inserted into the left ventricle cavity 3 at a more suitable location 34.

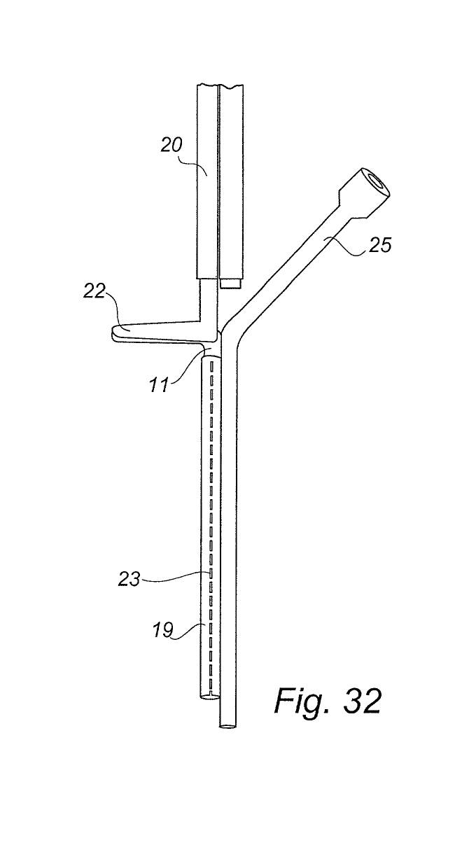

In other cases it might be advantageous not to put in a T-shaped conduit in the coronary artery 1. This might be the case, for instance, when the opening 30 in the artery 1 for anatomical reasons has to be done very close to the occlusion 6 in the artery 1, as illustrated in FIG. 31. In these cases there is no room for an extension of the second part 10 of the conduit in the direction of the occlusion 6. The new device 8 might also be used in such cases in another embodiment of the device 8 extending only in one direction in the coronary artery 1. The same insertion handle 20 may be used, now only with one handle arm 22, as shown in FIG. 32. The device 8 will still have the flexible part 11 between the first part 9 and the second part 10 of the conduit, which flexible part 11 permits the conduit to make the curve into the artery 1. The system for insertion is otherwise identical as well as the mode of operation.

FIG. 31 depicts the finished conduit in the case of a straight conduit rather than a T-shaped conduit. The disadvantage of the straight design is that the opening 30 in the artery 1 is not covered with the wall 12 of the second part 10 of the conduit and remains open and might cause a bleeding. The artery section proximal to the occlusion 6 will have to be closed by means of a clip or a ligature 35 to prevent bleeding in such cases.

It should be emphasised that the preferred embodiment described herein is in no way limiting and that many alternative embodiments are possible within the scope of protection defined by the appended claims.

What is claimed is:

1. A device for providing a supplemental flow of blood from the left ventricle of a heart to a coronary artery thereof in order to bypass an occlusion in said coronary artery, comprising a conduit having a first part to be positioned through the left ventricle wall into the left ventricle cavity and a second part to be positioned in said coronary artery, said first and second parts having longitudinal axes which are at an angle to each other, said conduit further having a flexible part forming a connection between the first part and the second part, whereby a channel is provided from a first open end of the first part to a first open end of the second part.

2. The device according to claim 1, wherein said conduit has a contracted state and a dilated state and is expandable from the contracted state to the dilated state.

3. The device according to claim 2, wherein the first and second parts of the conduit are separately expandable.

4. The device according to claim 1, wherein the flexible part allows movement of the second part along its longitudinal axis relative to the first part.

5. The device according to claim 1, wherein the flexible part allows varying the angle between the longitudinal axes of the first part and the second part.

6. The device according to claim 1, wherein the conduit comprises a stabilising means between the first part and the second part, said stabilising means substantially preventing movement of the first part along its longitudinal axis relative to the second part.

7. The device according to claim 6, wherein said stabilising means further prevents the second part of the conduit from rotating around its longitudinal axis.

8. The device according to claim 6, wherein the stabilising means comprises several rings substantially adjoining the periphery of the conduit.

9. The device according to claim 6, wherein the stabilising means comprises a coil.

10. The device according to claim 6, wherein the stabilising means comprises bars on opposite sides of the conduit.

11. The device according to claim 1, wherein the second part of the conduit extends from its first open end on one side of the connection between the first part and the second part to a second end on an opposite side of the connection between the first part and the second part.

12. The device according to claim 11, wherein the connection between the first part and the second part is closer to said second end of the second part than to said first open end thereof.

13. The device according to claim 1, wherein the first part has a second, open end and the second part has a second, open end, and wherein the flexible part connects these open ends, thereby forming said channel from the first open end of the first part to the first open end of the second part.

14. The device according to claim 1, wherein the first part comprises means for anchoring the device inside the left ventricle cavity of the heart.

15. The device according to claim 14, wherein the means for anchoring comprises an umbrella.

16. The device according to claim 14, wherein the means for anchoring comprises a cage.

17. The device according to claim 1, wherein the first and second parts comprise a memory material.

18. An introducer for a device for providing supplemental flow of blood from the left ventricle of a heart to a coronary artery thereof, said device comprising a conduit having a first part to be positioned through the left ventricle wall into the left ventricle cavity and a second part to be positioned in said coronary artery, said first and second parts having longitudinal axes which are at an angle to each other, said conduit further having a flexible part forming a connection between the first part and the second part, said introducer comprising a first introducing means for said first part of the conduit and a second introducing means for said second part of the conduit, said second introducing means comprising an L-shaped element for moving the second part of the conduit in relation to the first part of the conduit and utilising the flexible part of the conduit in order to introduce the second part of the conduit into the coronary artery when the first part is in place.

19. The introducer according to claim 18, wherein the first and the second introducing means comprise restraining means for keeping the first and second parts of the conduit in a contracted state, said first and second introducing means further comprising releasing means for releasing the first and second parts of the conduit from their contracted state.

20. The introducer according to claim 19, wherein the restraining means of the first introducing means comprises a tube enclosing the first part of the conduit in a contracted state, said tube having a longitudinal perforation, which is breakable for releasing the first part of the conduit from the contracted state into an expanded state.

21. The introducer according to claim 20, wherein said releasing means for releasing the first part from its contracted state comprises a balloon for breaking the perforation of the tube.

22. The introducer according to claim 19, wherein the first introducing means comprises a catheter, on which the restraining means is attached, for manoeuvring the introduction of the first introducing means.

23. The introducer according to claim 18, wherein the second introducing means comprises two L-shaped elements, said two elements being connected to form a T-shaped element, said T-shaped element being divisible into the two L-shaped elements for retraction of the L-shaped elements after introduction of the device.

24. A method for bypassing an occlusion in a coronary artery by providing a supplemental flow of blood from the left ventricle of a heart to said coronary artery by means of a device comprising a conduit having a first part and a second part, said first and second parts having longitudinal axes which are at an angle to each other, said conduit further having a flexible part connecting the first part and the second part, whereby a channel is provided from a first open end of the first part to a first open end of the second part, said method comprising the steps of
making an incision in said coronary artery distal to the occlusion,
creating a channel in a heart wall into the left ventricle cavity,
inserting the first part of the conduit into the channel by means of a first introducing means,
inserting the second part of the conduit through said incision into the coronary artery by means of the second introducing means,
and retracting said first and second introducing means.

25. The method according to claim 24, wherein the step of creating the channel is performed by means of a puncture needle, and the method further comprises the step of introducing a guide wire through the puncture needle for leading the first introducing means to the channel in the heart wall.

26. The method according to claim 25, further comprising the step of moving the second part antegrade into the coronary artery and then retrograde in the coronary artery in order to place it properly therein.

27. The method according to claim 24, wherein the device is expandable from a contracted state to an expanded state, and the method further comprises the step of expanding the device from the contracted state to the expanded state when the device has been properly placed in the coronary artery.

28. The method according to claim 24, further comprising the step of releasing the contraction of the first part of the conduit by inflating a balloon of the first introducing means to break a restraining means in the first introducing means.

29. The method according to claim 24, further comprising the step of blocking the blood flow in the coronary artery proximal to the second part of the conduit.

30. The method according to claim 24, wherein the puncture needle is inserted through a back wall of the artery into the left ventricle cavity so as to create a channel from the left ventricle cavity to the coronary artery.
